(19) United States
(12) Patent Application Publication
(10) Pub. No.: US 2022/0064092 A1
(43) Pub. Date: Mar. 3, 2022

(54) METHYLATION PROCESS

(71) Applicant: FOOD FOR FUTURE S.R.L. SOCIETÀ BENEFIT, MILANO (IT)

(72) Inventor: Guglielmo BUONAMICI, SAN GIULIANO TERME (Pisa) (IT)

(21) Appl. No.: 17/424,275

(22) PCT Filed: Jan. 21, 2020
(86) PCT No.: PCT/IB2020/050439

§ 371 Date: Jul. 20, 2021

(30) Foreign Application Priority Data
Jan. 21, 2019 (IT) 102019000000865

(51) Int. Cl. C07C 41/16

(52) U.S. Cl. CPC C07C 41/16

(57) ABSTRACT

A methylation process is provided, including mixing resveratrol, at least one methyl-group donor, for example trimethylglycine, folic acid, and wherein the mixing of resveratrol with trimethylglycine and folic acid is so that the least one methyl-group donor carries out the methylation of resveratrol by yielding methyl groups thanks to the folic acid.

The present invention concerns a methylation process of the type specified in the preamble of the first claim.

In particular, the invention concerns a methylation process, conveniently present in plant extracts, in pterostilbene. For example, the process described here enables resveratrol methylation that is naturally present in wine or in certain plant extracts. Resveratrol (3,5,4′-trihydroxy-trans-stilbene) is a polyphenol, one of the phytoalexins naturally produced by some plant species in defence of pathogens such as bacteria or fungi.

Resveratrol (3,5,4′-trihydroxy-trans-stilbene) is a polyphenol, one of the phytoalexins naturally produced by some plants—it is present in grape skin for example—in defence against pathogens such as bacteria or fungi. It is attributed with possible anticancer, anti-inflammatory, and blood thinning actions, which can limit the onset of thrombotic plaques. Resveratrol has applications in the medical field thanks to its multiple effects.

Resveratrol is in fact particularly active as a skin anti-ageing treatment, an antioxidant, and an anti-inflammatory.

In detail, resveratrol, being a phytoalexin produced by plants for protective purposes, protects against viruses, bacteria, fungi, and environmental stresses. It also has a powerful anti-oxidant and anti-ageing action that, in human beings, makes it possible to delay ageing and the expression of the traits of ageing. The antioxidant action of resveratrol is owed to its ability to inhibit lipid peroxidation of low-density lipoproteins and also by its action upstream of the reaction and its ability to deactivate copper as a catalyst.

The described prior art comprises some significant drawbacks. In particular, resveratrol has a reduced bioactivity (about 20%) that results in an extremely reduced action compared to its real efficacy.

In addition, resveratrol has a lower resistance to degradation and elimination and a high rate of glucuronidation and sulphation, defining a very limited half-life for the resveratrol.

Finally, it should be noted that the amount of resveratrol in wine is small and, therefore, a correct dosage would require the intake of 3 to 4 litres of wine per day with obvious damage to health.

In this context, the technical task underlying the present invention is to devise a methylation process capable of substantially overcoming at least some of the above-mentioned drawbacks.

Within said technical task it is an important purpose of the invention to obtain a methylation process able to increase the quantity of pterostilbene resulting from resveratrol methylation.

The technical task and the specified purposes are achieved by means of a methylation process as claimed in appended claim 1. Examples of preferred embodiments are described in the dependent claims.

In the present document, the measures, values, shapes and geometric references (such as perpendicularity and parallelism), when associated with words like “almost” or other similar terms such as “approximately” or “substantially”, are to be understood as except for measurement errors or inaccuracies owing to production and/or manufacturing errors and, above all, except for a slight divergence from the value, measure, shape, or geometric reference with which it is associated. For example, if such terms are associated with a value, they preferably indicate a divergence of not more than 10% of the same value.

The measurements and data provided in the present text are to be considered as performed in ICAO International Standard Atmosphere (ISO 2533), unless otherwise indicated.

The methylation process is aimed at the formation of pterostilbene for resveratrol methylation.

It should be noted that with the methylation process, i.e. with the methylation, the resveratrol is not transformed into pterostilbene, but rather the methylation process, described here, results in a greater production of pterostilbene. Therefore, expressions such as “methylation of resveratrol into pterostilbene” identify the production of pterostilbene owing to the presence of resveratrol in accordance with the process described here.

In particular, the process performs a resveratrol methylation, resulting in the increased production of pterostilbene. Because the process induces additional resveratrol methylation, it results in increased production of pterostilbene (also called trans-3,5-dimethoxy-4-hydroxystilbene), a compound chemically similar to resveratrol and belonging, like resveratrol, to the group of phytoalexins synthesised by plants in response to helical agents and stressful situations.

Pterostilbene is extremely similar to resveratrol (to be precise, it is a stilbenoid like resveratrol) and, therefore, able to perform the same actions: scavenger, immunostimulant, adaptogenic, cardiovascular protection, cancer prevention, hypoglycaemic, hypotriglycerising, antihypertensive, homocysteine containing, hypocholesterolemic, and antithrombotic.

Pterostilbene is a polyphenol that is biochemically derived from the methylated form of resveratrol and has a bioactivity of 80% and a bioactive action more than 200 times greater than that of resveratrol. It is also more widely diffused within the cells and has a longer half-life than resveratrol because it is more resistant to degradation and elimination.

The methylation process comprises resveratrol.

Conveniently, it comprises Polygonum Cuspidatum extracts comprising resveratrol. Alternatively, the methylation process may comprise other plant extracts comprising resveratrol, such as Vitis vinifera, Vaccinium vitis-idaea, Vaccinium myrtillus, Helleborus niger, and Pterocarpus marsupium.

The methylation process may comprise a methyl-group donor, i.e. may yield one or more methyl groups to make the methylation of resveratrol into pterostilbene possible.

The methyl-group donor can be selected from one or more of the following: trimethylglycine, DNA-methyltransferase.

In detail, the methylation process comprises trimethylglycine.

More specifically, the methylation process comprises Beta vulgaris L. extracts (hereinafter simply Beta vulgaris) comprising trimethylglycine and/or Stachys tuberifera extracts comprising trimethylglycine. Trimethylglycine and, to be precise, said extracts act in the methylation reaction of plant DNA as an additional donor of methyl groups by inhibiting the Hill or Blacman reaction.

Alternatively or in addition, the methylation process comprises Arabidopsis thaliana extracts containing DNA-methyltransferase. The DNA-methyltransferase and, to be precise, said extracts carry out the transfer and insertion, with covalent bonds, of methyl groups to the DNA wherein the DRM2, MET1, and CMT3 enzymes also intervene.

The content of trimethylglycine and, in particular, Beta vulgaris extracts is at least equal to, substantially, 100%, more specifically to 250%, and even more specifically to 1000% of the content of resveratrol and, in particular, of said extracts (preferably Polygonum cuspidatum) comprising resveratrol.

Alternatively or in addition, the methylation process comprises Chenopodium quinoa extracts comprising trimethylglycine.

The methylation process preferably comprises Beta vulgaris extracts comprising trimethylglycine and Chenopodium quinoa extracts comprising trimethylglycine.

The content of trimethylglycine and, in particular, Chenopodium quinoa extracts is less than that of the Beta vulgaris extracts. More specifically, it is significantly less than 50%, even more specifically 10% of the content of Beta vulgaris extracts. The content of Chenopodium quinoa extracts substantially ranges between 15% and 5% of the content of Beta vulgaris extracts.

As an alternative, or in addition, the methyl-group donors can be Saccharomyces cerevisiae.

The methylation process comprises folic acid and, more specifically, Medicago sativa extracts comprising folic acid.

The content of trimethylglycine and, in particular, Medicago sativa extracts is less than that of the Beta vulgaris extracts and, conveniently, of Chenopodium quinoa extracts. More specifically, it is substantially less than 15%, even more specifically 5% of the content of Beta vulgaris extracts. The content of trimethylglycine and, in particular, of Medicago sativa extracts substantially ranges between 2% and 1% of the content of Beta vulgaris extracts.

The methylation process may comprise methyl alcohol conveniently donating methyl groups.

Methyl alcohol can be extracted via the dry distillation of wood and from the residual by-product (pyroligneous acid).

The methyl alcohol content substantially ranges between 200% and 15% and, more specifically, between 100% and 33% of the content of the methyl-group donors, more specifically, of trimethylglycine and, to be precise, of Beta vulgaris extracts. For example, in one litre or kilogram of product (such as, for example, a food such as a dairy product, pasta, an alcoholic or non-alcoholic drink, or a herbicide) the methylation process may comprise the following contents of extracts of: Polygonum cuspidatum substantially ranging between 0.1 g and 5 g, more specifically, between 0.2 g and 3 g, and, to be precise, 0.3 g and 1.5 g; Medicago sativa substantially ranging between 0.01 g and 2 g, more specifically 0.02 g and 1 g, and, to be precise, 0.03 g and 0.6 g; Chenopodium quinoa substantially ranging between 0.01 g and 2 g, more specifically between 0.02 g and 1 g, and, to be precise, between 0.1 g and 5 g, more specifically between 0.2 g and 3 g, and, to be precise, between 0.3 g and 1.5 g; Beta vulgaris, substantially ranging between 1 g and 100 g, more specifically between 2 g and 50 g, and, to be precise, between 2 g and 50 g; and, conveniently, methylic acid substantially ranging between 1 g and 100 g, more specifically between 2 g and 30 g, and, to be precise, 2 g and 10 g.

Said extracts are mixed together subsequently or, preferably, before their incorporation into said product.

The methylation process is mainly carried out by the methyl-group donor (e.g. trimethylglycine or DNA-methyltransferase) and by folic acid, which makes at least part, conveniently all, of the methyl groups present in trimethylglycine available, enabling the methylation of said resveratrol into pterostilbene. More specifically, it is carried out by the action of the methyl-group donors (e.g. trimethylglycine or DNA-methyltransferase) with the methionine synthase enzyme and with vitamin B12 as coenzyme, which perform the action of reducing 5-methyltetrahydrofolate (MTHF) to methyltetrahydrofolate, which, in turn, provides the methyl group necessary for the methylation of resveratrol and the formation of pterostilbene.

To be precise, the methylation process comprises the mixing of resveratrol with at least the methyl-group donor (e.g. trimethylglycine or DNA-methyltransferase) and folic acid so that the donor carries out the resveratrol methylation thanks to the assistance of the folic acid that, as catalyst, favours the release of at least part, and preferably all of, the methyl groups present in at least the methyl-group donor and necessary for the methylation of resveratrol into pterostilbene. More specifically, the methylation process comprises mixing extracts (such as Polygonum cuspidatum extracts) comprising resveratrol with at least the Medicago sativa extracts comprising folic acid and at least either: Beta vulgaris extracts, comprising trimethylglycine, or Chenopodium quinoa extracts, comprising trimethylglycine. More specifically, the methylation process comprises mixing extracts (such as Polygonum cuspidatum extracts) comprising resveratrol with at least Medicago sativa extracts comprising folic acid, Beta vulgaris extracts comprising trimethylglycine, and Chenopodium quinoa extracts comprising trimethylglycine. Preferably, the methylation process comprises mixing resveratrol with, in addition to the above-mentioned components, methyl alcohol that, by yielding methyl groups, further favours the transformation of resveratrol into pterostilbene.

The methylation process described above can be applied to the preparation of additives to be added to beverages (whether alcoholic or not) and/or foods (such as flour and dairy products), and can preferably be used to produce said beverages and/or foods.

For example, it can be used for producing an alcoholic beverage and, in particular, an additive to be added to an alcoholic beverage and, preferably, to a wine. The methylation process can, thus, be part of a method for producing an alcoholic beverage conveniently comprising the production of an additive.

The method for producing an alcoholic beverage comprises the methylation process described above and an addition process wherein the additive is obtained by adding: cycloastragenol (conveniently Astragalus membranaceus extracts comprising said cycloastragenol); cynarine and cynaropicrin (Cynara scolymus extracts comprising said cynarine and cynaropicrin); and folic acid (Medicago sativa extracts comprising said folic acid) to at least the pterostilbene and/or resveratrol (of said methylation process).

In said addition process, at least one of and, more specifically, all of: chlorogenic acid (conveniently, Moringa oleifera extracts containing said chlorogenic acid); isoflavones and said coenzymes Q10 (conveniently Glycine max extracts comprising said isoflavones and said coenzymes Q10); zeatin and quercetin (conveniently Moringa oleifera extracts containing zeatin and quercetin); kaempferol (conveniently one or more of: Moringa Oleifera, Aloe vera, Coccinia grandis, Cuscuta chinensis, Euphorbia pekinensis, Glycine max, Hypericum perforatum, Salvia rosmarinus, Sambucus nigra, Toona sinensis, and Ilex extracts) can be added.

The contents, calculated per litre of alcoholic beverage, of said extracts may be: Polygonum cuspidatum substantially ranging between 0.2 g/l and 5 g/l; Astragalus substantially ranging between 2 g/l and 30 g/l; Moringa oleifera substantially ranging between 0.1 g/l and 0.5 g/l; Cynara substantially ranging between 0.1 g/l and 2 g/l; Medicago sativa substantially ranging between 0.15 g/l and 2 g/l; and Glycine max substantially ranging between 0.15 g/l to 2 g/l. These contents can be used individually based on the extracts introduced.

Finally, the method for producing an alcoholic beverage comprises a process for mixing the additive to the alcoholic beverage and, in particular, to a wine. In the case of the production of a cosmetic, the methylation process can be part of a method for preparing a cosmetic.

The method for preparing a cosmetic thus comprises the methylation process and, conveniently, a process for supplementing at least the pterostilbene and/or resveratrol (of said methylation process), and a cosmetic preparation, with: betaine (conveniently Beta vulgaris extracts comprising betaine), folic acid (conveniently Medicago sativa extracts comprising folic acid), and trimethylglycine (conveniently

Beta vulgaris extracts comprising trimethylglycine).

For 100 g of cosmetic product, the content of the extracts may be: Beta vulgaris substantially ranging between 4 g and 8 g; Medicago sativa approximately ranging between 4 g and 8 g; and at least one of: Polygonum cuspidatum, substantially ranging between 0.2 g and 0.8 g, or Vitis vinifera, substantially ranging between 0.4 g and 0.8 g.

In some cases, the supplementing process may involve the addition of coenzyme Q10, conveniently contained in Medicago Sativa extracts; and at least one of: TA-65, conveniently contained in Astragalus membranaceus extracts; mucopolysaccharides, conveniently contained in Fucus vesiculosus extracts; arthrospira, conveniently contained in alga Arthrospira fusiformis extracts; and hyaluronic acid, conveniently contained in Tremella fuciformis extracts. In detail: It should be noted that per 100 g of cosmetic product, a content of: Astragalus membranaceus extracts, substantially ranging between 0.2 g and 1 g; Fucus vesiculosus extracts, substantially ranging between 0.2 g and 0.6 g; alga Arthrospira fusiformis extracts, substantially ranging between 2 g and 4 g; Tremella fuciformis extracts, substantially ranging between 4 g and 6 g, may be used.

The methylation process described above can be applied in the preparation of a human nutraceutical supplement, preferably in pill or sachet form.

The method for preparing a human nutraceutical supplement thus comprises the methylation process and, conveniently, a process for adding: muirapuamine alkaloid (Muira puama marapuama extracts, conveniently derived from roots, branches, and/or bark, comprising muirapuamine alkaloid); damiana (more specifically, Turnera aphrodisiaca Willd. ex Schult extracts, conveniently derived from leaves and/or roots, comprising damiana); icariin (Epimedium grandiflorum extracts, also called Epimedium macranthum var. violaceum (C. Morren & Decne.), comprising icariin and preferably derived from dried leaf); Eurycomanone (more specifically, Eurycoma longifolia Jack extracts, preferably derived from the root, comprising Eurycomanone); one or more of: genistein and daidzein and arginine (in Glycine max extracts, usually derived from seeds, comprising genistein and daidzein and arginine); at least one of: coenzyme Q10, Vitamin E, magnesium, calcium, phosphorus (more specifically, Triticum turgidum extracts, preferably from wheat germ, comprising coenzyme Q10, Vitamin E, magnesium, calcium, phosphorus); at least one of: pterostilbene, kino secretion, tannins, butein (more specifically, Butea frondosa extracts, preferably from the root, comprising pterostilbene, kino secretion, tannins, butein); at least one of: pterostilbene, organic acids, pectins, tannins, myrtillin, anthocyanins, vitamins A, C, and B (more specifically, Vaccinium myrtillus extracts, preferably the fruit, comprising pterostilbene and, preferably, organic acids, pectins, tannins, myrtillin, anthocyanins, vitamins A, C, and B); Plygonum cuspidatum extracts; and, in some cases, excipients, to at least the pterostilbene and/or resveratrol (of said methylation process).

To be precise, a content of said extracts may be added during the addition process: Muira puama marapuama substantially ranging between 15% and 2%; Turnera aphrodisiaca substantially ranging between 15% and 2%; Epimedium substantially ranging between 15% and 2%; Eurycoma longifolia substantially ranging between 15% and 2%; Glycine max substantially ranging between 15% and 2%; Triticum turgidum substantially ranging between 5% and 1%; Butea frondosa substantially ranging between 15% and 2%; Vaccinium myrtillus substantially ranging between 15% and 2%; Polygonum cuspidatum substantially ranging between 15% and 2%; Vitis vinifera approximately ranging between 15% and 2%; Beta vulgaris substantially ranging between 15% and 2%.

Optionally, during the addition process, one or more of: catuabines, conveniently A, B, C and D; and/or cinchonine and, more specifically, Erythroxylum catuaba extracts, preferably of the bark, comprising catuabines and/or cinchonine, can be added. The content of said Erythroxylum catuaba extracts substantially ranges between 5% and 1%.

Optionally, during the addition process, yohimbine and, more specifically, Pausinystalia johimbe (K. Schum.) extracts, preferably the bark, comprising yohimbine, can be added. The content of said Pausinystalia johimbe extracts substantially ranges between 5% and 1%.

Optionally, beta-Sitosterol may be added during the addition process, in Serenoa repens extracts, preferably of dried fruits, comprising beta-Sitosterol. The content of said Serenoa repens extracts substantially ranges between 5% and 1%.

Optionally, during the addition process, one or more of: cucurbitins, delta sterols, phytosterols, plant globulins, vitamin F and E; and, more specifically, Cucurbita pepo L. extracts (hereinafter referred to simply as Cucurbita pepo), preferably from seeds, comprising cucurbitins, delta sterols, phytosterols, plant globulins, vitamin F and E, can be added. The content of said Cucurbita pepo extracts is substantially less than 3%.

Optionally, during the addition process one or more of: iron, calcium, silicon, magnesium, phosphorus, vitamin A, C, and K, formic and gallic acid, chlorophyll, tannin, carotene and histamine; in Urtica dioica L. extracts (hereinafter referred to simply as Urtica dioica), preferably from leaves, comprising iron, calcium, silicon, magnesium, phosphorus, vitamin A, C, and K, formic and gallic acid, chlorophyll, tannin, carotene, and histamine, can be added. The content of said Urtica dioica extracts is substantially less than 3%.

The content of said Medicago sativa extracts may be substantially less than 3%. The methylation process described above can be applied in preparing a rodenticide, i.e. a pesticide used to kill or eliminate the presence or action of rodents and, to be precise, mice, i.e. a mouse poison.

The method for preparing a rodenticide thus comprises the methylation process and, conveniently, a process of mixing extracts of: Aesculus hippocastanum, Prunus laurocerasus, Digitalis purpurea, Melilotus officinalis, Ricinus communis, Salix alba, egg albumin, and Peniccillum chrysogenum; and, preferably, an attractive compound for rodents, with at least pterostilbene and/or resveratrol (of said methylation process).

More specifically, during the mixing process, a content of extracts of: Aesculus hippocastanum substantially ranging between 10% and 5%; Prunus laurocerasus substantially ranging between 1% and 0.5%; Digitalis purpurea substantially ranging between 30% and 25%; Melilotus officinalis substantially ranging between 5% and 1%; Ricinus communis substantially less than 0.1%; Salix alba substantially ranging between 5% and 1%; egg albumin substantially ranging between 5% and 1%; and Penicillum chrysogenum substantially ranging between 8% and 3%, can be added.

Optionally, Atropa belladonna extracts can be added during the mixing process in a quantity conveniently, approximately ranging between 5% and 1%.

Optionally, Fucus vesiculosus extracts can be added during the mixing process in a quantity conveniently, approximately ranging between 20% and 10%.

Optionally, Fucus vesiculosus extracts can be added during the mixing process in a quantity conveniently, approximately ranging between 20% and 10%.

Optionally, Penicillum brefeldianum extracts can be added during the mixing process in a quantity conveniently, approximately ranging between 8% and 3%.

Optionally, Penicillum notatum extracts can be added during the mixing process in a quantity conveniently, approximately ranging between 8% and 3%.

The methylation process according to the invention achieves important advantages. In fact, in nature the formation of pterostilbene results from a “natural” methylation and is modest in quantity and almost without health effects. The method described here performs a powerful methylation action on the resveratrol itself giving rise to a greater quantity of pterostilbene to the measure of 20% that, thanks to this, assumes the real health role that has been outlined.

In addition, unlike the well known methylation processes, the methylation process described here makes it possible to carry out the methylation and, therefore, the formation of pterostilbene, resulting from the methylation of resveratrol, i.e. an active ingredient that, as described above, increased the beneficial capacities.

Resveratrol methylation, which produces more pterostilbene, results from the presence of methyl-group donors (e.g. trimethylglycine or DNA-methyltransferase), i.e. strong methylating agents capable of yielding methyl groups (CH₃) to resveratrol and, to be precise, performing the synthesis and donation of methyl groups.

In particular, its action, associated with the methionine synthase enzyme, with vitamin B12 as a coenzyme and folic acid, carries out the resveratrol methylation. They reduce 5-methyltetrahydrofolate (MTHF) to methyltetrahydrofolate thus supplying the methyl groups necessary for the formation of pterostilbene.

The donor and, more specifically, trimethylglycine increases the quantity of S-Adenosyl methionine (SAM) catalysing in the resveratrol methylation reactions and, possessing a chemically reactive methyl group, which extends the action to other molecules through trans-methylation reactions.

It should be noted that methylation is made possible by the presence of the donor and, more specifically, of trimethylglycine and SAM, which facilitate this process. More specifically, trimethylglycine assists the action of S-Adenosyl methionine (SAM) and of the methionine synthase enzyme that intervenes in the methylation and that, together with vitamin B12 as coenzyme and folic acid, intervenes in the reduction of 5-methyltetrahydrofolate to methyltetrahydrofolate that, in turn, provides the methyl group necessary for methylation.

The methylation of resveratrol into pterostilbene is also helped by methylic acid that, by making available a good number of methyl groups, facilitates the formation of pterostilbene.

Finally, it should be noted that the methylation of resveratrol into pterostilbene is assisted by the particular relationship between the various active ingredients and, therefore, between the extracts.

We should emphasise that the extracts used are only plant extracts.

Advantages can be found, for example, in the production of an alcoholic beverage where, by interacting with the alcohol content and/or the various active ingredients present in said alcoholic beverage and, in particular, in wine, it increases the properties of this drink/wine, for example the antioxidant, cardiovascular, DNA regeneration, anticancer, and anti-flogistic (anti-inflammatory) properties, etc.

Moreover, important advantages can be found in the cosmetic preparation where the use of the methylation process makes it possible to give the cosmetic product strong scavenger, compacting, anti-inflammatory, moisturising, toning effects. It is characterised by a strong anti-skin cancer action and a high synthesis capacity of products such as collagen, elastin, hyaluronic acid, carbohydrates, and proteins and an increase in cellular longevity.

In addition, the innovative method for preparing a nutraceutical supplement makes it possible to produce a nutraceutical supplement able to perform, in terms of stimulating the male genital apparatus, an important action thanks to the synergistic action of rebalancing the nervous system with consequent increase in libido/stimulation (thanks to the extracts of Turnera aphrodisiaca Willd. ex Schult, Muira puama marapuama, Triticum turgidum, Erythroxylum catuaba, Erythroxylum, Eurycoma longifolia Jack, and Glycine max) and thanks to an improvement in the cardiovascular system thanks to the active ingredients present in the extracts of Turnera aphrodisiaca Willd. ex Schult, Epimedium grandiflorum icariin, Glycine max, Polygonum cuspidatum, Vitis vinifera, and Butea.

In addition, the rodenticide that can be obtained with the method for preparing a rodenticide is completely biological since its toxic agents are only of plant extraction; and thanks to the high activity of the rodenticide, which is guaranteed by the particular combination of phytocomplexes and mycetes as demonstrated by studies by the inventor. It has a strong cardiovascular-respiratory action that leads to the rodent's quick death.

The invention is subject to variations falling within the scope of the inventive concept defined by the claims. In this context all the details may be replaced with equivalent elements and the materials, shapes, and dimensions may be as desired.

It should be noted that the extracts presented in this document, unless otherwise indicated, can be obtained through a drying and titration process of the powders or by solvent extraction.

In some cases, they can be obtained by Soxhlet reflux extraction and, in particular, with ultrasound or supercritical gas, and the addition of sulphur dioxide as the solvent. Preferably, these extracts are obtained through the Soxhlet reflux method with methanol. This method comprises the fermentation and hydrolysis of the part of the plant (for example fruit, flower or leaf) from which the extracts are to be obtained, with the addition of yeast, appropriately Saccharomycetaceae (such as Saccharomyces cerevisiae) and preferably operating at pH 7 for about 4 days; evaporation and centrifugation to remove the solvent from the solution obtained above; Soxhlet extraction; methanol removal by boiling-point heating, and extraction thereof by evaporation.

1. A methylation process comprising mixing resveratrol;
at least one methyl-group donor; and
folic acid; and
the mixing of said resveratrol with said methyl-group donor and said folic acid so that said folic acid makes at least part of the methyl groups present in said methyl-group donor available so that the methyl-group donor carries out the methylation of said resveratrol producing pterostilbene.

2. The methylation process according to claim 1, wherein said methyl-group donor is selected from: trimethylglycine and DNA-methyltransferase.

3. The methylation process according to claim 2, wherein said methyl-group donor comprises at least either: Beta vulgaris extracts, comprising said trimethylglycine; or Chenopodium quinoa extracts, comprising said trimethylglycine.

4. The methylation process according to claim 3, wherein said Beta vulgaris extracts comprise said trimethylglycine and said Chenopodium quinoa extracts comprise said trimethylglycine; and wherein the content of said Chenopodium quinoa extracts substantially ranges between 15% and 5% of the content of said Beta vulgaris extracts.

5. The methylation process according to claim 1, wherein Medicago sativa extracts comprising said folic acid are included during the mixing.

6. The methylation process according to claim 5, wherein the content of said Medicago sativa extracts substantially ranges between 2% and 1% of said content of said Beta vulgaris extracts.

7. The methylation process according to claim 1, wherein the methylation comprises methyl alcohol donating methyl groups.

8. The methylation process according to claim 7, wherein the content of said methyl alcohol substantially ranges between 100% and 33% of said content of said Beta vulgaris extracts.

9. The methylation process according to claim 1, wherein Polygonum cuspidatum extracts comprising said resveratrol are included during the mixing.

10. A method for producing an alcoholic beverage comprising said methylation process according to claim 1; an addition process wherein an additive of said alcoholic beverage is obtained by adding cycloastragenol, cynarine, cynaropicrin, and folic acid to said pterostilbene resulting from said methylation process; and a process for mixing said additive to said alcoholic beverage.

11. The method for producing an alcoholic beverage according to claim 10, wherein Astragalus membranaceus extracts, comprising said cycloastragenol, Cynara scolymus extracts, comprising said cynarine and said cynaropicrin, and Medicago sativa extracts, comprising folic acid, are added during the addition process.

12. A method for preparing a cosmetic comprising said methylation process according to claim 1; and a process for supplementing said pterostilbene, resulting from said process for methylating a cosmetic preparation, with: betaine, folic acid, and trimethylglycine.

13. The method for preparing a cosmetic according to claim 1, wherein Beta vulgaris extracts, comprising said betaine; Medicago sativa extracts, comprising said folic acid; and Beta vulgaris extracts, conveniently comprising said trimethylglycine, are added during said supplementing process.

14. A method for preparing a human nutraceutical supplement comprising said methylation process according to claim 1; and a process for adding Muira puama marapuama extracts, Turnera aphrodisiaca Willd extracts, Epimedium grandiflorum extracts, Eurycoma longifolia Jack extracts, Glycine max extracts, Triticum turgidum extracts, Butea frondosa extracts, Vaccinium myrtillus extracts, and Plygonum cuspidatum extracts to said pterostilbene, resulting from said process.

15. A method for preparing a rodenticide comprising said methylation process according to claim 1; and a mixing process wherein Aesculus hippocastanum extracts, Prunus laurocerasus extracts, Digitalis purpurea extracts, Melilotus officinalis extracts, Ricinus communis extracts, Salix alba extracts, egg albumin extracts, and Peniccillum chrysogenum extracts are added to said pterostilbene, resulting from said methylation process.

16. The methylation process according to claim 1, wherein said methyl-group donor comprises trimethylglycine.

17. The methylation process according to claim 1, wherein said methyl-group donor comprises DNA-methyltransferase.
